(19) United States
(12) Patent Application Publication
(10) Pub. No.: US 2024/0400967 A1
(43) Pub. Date: Dec. 5, 2024

(54) Cell Culture Device And Calibration Method

(71) Applicant: Terumo Kabushiki Kaisha, Tokyo (JP)

(72) Inventors: Itaru OKUBO, Kanagawa (JP); Liming LIU, Kanagawa (JP)

(21) Appl. No.: 18/799,401

(22) Filed: Aug. 9, 2024

Related U.S. Application Data
(63) Continuation of application No. PCT/JP2023/009203, filed on Mar. 10, 2023.

(30) Foreign Application Priority Data
Mar. 28, 2022 (JP) 2022-051961

(51) Int. Cl. C12M 1/34; C12M 1/00

(52) U.S. Cl. CPC C12M 41/00; C12M 27/18; C12M 41/36

(57) ABSTRACT

A cell culture device includes a cell culture circuit, a sensor that is disposed in the cell culture circuit and is configured to measure turbidity of a cell fluid, and a calibration unit. The calibration unit is configured to calculate, as a first concentration, a cell concentration of a culture medium that includes the cell fluid, to calculate, as a second concentration, a cell concentration of a culture medium that includes no cells, and to create a calibration curve for a measurement value of the sensor and the turbidity of the cell fluid by associating the first concentration with a first measurement value for the culture medium that includes the cell fluid and associating the second concentration with a second measurement value for the culture medium that includes no cells.

### CROSS-REFERENCE TO RELATED APPLICATIONS

The present application is a continuation of the International Patent Application No. PCT/JP2023/009203 filed Mar. 10, 2023, which designated the U.S. and claims the benefit of priority from Japanese Patent Application No. JP2022-051961 filed Mar. 28, 2022. The entire disclosures of the above-identified are incorporated herein by applications reference.

### FIELD

The present disclosure relates to a cell culture device and a calibration method for the cell culture device.

### BACKGROUND

A cell culture device for culturing cells is disclosed in JP 2015-50983. The cell culture device includes a turbidity sensor that is used to measure the cell concentration of a culture medium. The turbidity sensor measures, for example, transmitted light, scattered light, or a combination of transmitted light and scatter light of a light applied to the culture medium. The greater the cell concentration, the greater the number of cells in the culture medium, increasing the turbidity.

Turbidity may be affected by cell size. For example, when comparing a culture medium A containing small cells with a culture medium B containing large cells, where both culture medium A and culture medium B includes the same number of cells, the light transmittance of the culture medium A will be higher than the light transmittance of the culture medium B. That is, for culture medium A and culture medium B having the same cell concentration, the turbidity of the culture medium A will be lower than the turbidity of the culture medium B. The relationship between the turbidity and the cell concentration may vary in response to cell type. Therefore, when a turbidity sensor is used to measure cell concentration, it is often necessary to set the relationship between the cell concentration and the turbidity in advance. For example, the relationship between the cell concentration and the turbidity may be set by a calibration curve.

Calibration work is often required to prepare the calibration curve. For example, it is often necessary to prepare two cell having different cell concentrations and attaching the turbidity sensors to the cell culture device only after the turbidity sensors measured the turbidity of the two cell fluids individually.

Since the cell fluid and culture medium are often discarded after the calibration work, there is a waste of the cell fluid and the culture medium. In addition, the conventional calibration work occurs outside of the cell culture device and also often requires various independent steps, which negatively impacts the convenience of using the cell culture device.

### SUMMARY

In at least one example embodiment, a cell culture device is provided. The cell culture device may include a cell culture circuit that is configured to circulate a culture medium in which a cell fluid is supplied, a sensor that is disposed in the cell culture circuit to measure a turbidity of the cell fluid, and a calibration unit that calibrates the measurement value of the sensor to a cell concentration of the culture medium. The sensor may be configured to measure, as a first measurement value, a turbidity of a first volume of the culture medium, where the first volume includes a cell fluid including a predetermined number of cells. The sensor may also be configured to measure, as a second measurement value, a second turbidity of a second volume of the culture medium, where the second volume of the culture medium is prepared by diluting the first volume of the culture medium with a volume of the culture medium that does not include cells. The calibration unit may calculate, as a first concentration, the cell concentration of first volume of the culture medium. The calibration unit may calculate, as a second concentration, the cell concentration of the second volume of the culture medium. The calibration unit may create a calibration curve between the measurement value of the sensor and the cell concentration by associating the first concentration with the first measurement value and by associating the second concentration with the second measurement value.

In at least one example embodiment, a calibration method for a cell culture device is provided. The cell culture device may include a cell culture circuit that circulates a culture medium that includes a cell fluid. The cell culture device may include a sensor that measures turbidity of the cell fluid. The sensor may be disposed in the cell culture circuit. The cell culture device may include a calibration unit that calibrates measurement values of the sensor to a cell concentration of the culture medium. The method may include causing the sensor to measure, as a first measurement value, a first turbidity of a first volume of the culture medium, where the first volume of the culture medium includes a cell fluid including a predetermined number of cells. The method may also include causing the sensor to measure, as a second measurement value, a second turbidity of a second volume of the culture medium, where the second volume of the culture medium is prepared by diluting the first volume of the culture medium with a volume of the culture medium that does not include cells. The method may include causing the calibration unit to calculate, as a first concentration, the cell concentration of the first volume of the culture medium. The method may include causing the calibration unit to calculate, as a second concentration, the cell concentration of the second volume of the culture medium. The method may include causing the calibration unit to create a calibration curve between the measurement value of the sensor and the cell concentration by associating the first concentration with the first measurement value and by associating the second concentration with the second measurement value.

In at least one example embodiment, it is possible to prepare a cell fluid in which the number of cells is known in advance and to perform calibration using a cell culture device without having to perform an operation outside the cell culture device. Accordingly, the efficiency of cell culturing is improved and also waste may be eliminated at least because cell fluid and culture medium do not need to be preemptively discarded.

### DRAWINGS

FIG. 1 is a schematic of an example cell culture device in accordance with at least one example embodiment.

FIG. 2 is a block diagram of an example arithmetic unit for use with a cell culture device, like the cell culture device illustrated in FIG. 1, in accordance with at least one example embodiment.

FIG. 3 is a schematic of an example sensor unit for use with a cell culture device, like the cell culture device illustrated in FIG. 1, in accordance with at least one example embodiment.

FIGS. 4A and 4B are schematics illustrating example connection states between a first circulation flow path and a dilution flow path of a sensor unit, like the sensor unit illustrated in FIG. 3, in accordance with at least one example embodiment.

FIG. 5 is a flowchart illustrating an example calibration method for a cell culture device, like the cell culture device illustrated in FIG. 1, in accordance with at least one example embodiment.

FIG. 6 is a graphical demonstration illustrating an example calibration curve in accordance with at least one example embodiment.

FIG. 7 is a schematic of another example sensor unit for use with a cell culture device, like the cell culture device illustrated in FIG. 1, in accordance with at least one example embodiment.

FIGS. 8A and 8B are schematics illustrating example connection states between a first circulation flow path and a dilution flow path of a sensor unit, like the sensor unit illustrated in FIG. 7, in accordance with at least one example embodiment.

FIG. 9 is a schematic of another example sensor for use with a cell culture device, like the cell culture device illustrated in FIG. 1, in accordance with at least one example embodiment.

FIG. 10 is a schematic of another example cell culture device in accordance with at least one example embodiment.

FIG. 11 is a schematic of another example sensor unit for use with a cell culture device, like the cell culture device illustrated in FIG. 10, in accordance with at least one example embodiment.

FIGS. 12A-12D are schematics illustrating example connection states of a first circulation flow path, a sensor flow path, and a concentration flow path of a sensor unit, like the sensor unit illustrated in FIG. 11, in accordance with at least one example embodiment.

FIG. 13 is a flowchart illustrating an example calibration method for a cell culture device, like the cell culture device illustrated in FIG. 10, in accordance with at least one example embodiment.

FIG. 14 is a schematic of another example sensor unit for use with a cell culture device, like the cell culture device illustrated in FIG. 10, in accordance with at least one example embodiment.

FIG. 15 is a schematic of another example sensor unit for use with a cell culture device, like the cell culture device illustrated in FIG. 10, in accordance with at least one example embodiment.

FIGS. 16A-16D are schematics illustrating connection states between a first circulation flow path, a sensor flow path, a concentration flow path, and a dilution flow path of a sensor unit, like the sensor unit illustrated in FIG. 15, in accordance with at least one example embodiment.

FIG. 17 is a flowchart illustrating another example calibration method for a cell culture device, like the cell culture device illustrated in FIG. 10, in accordance with at least one example embodiment.

### DETAILED DESCRIPTION

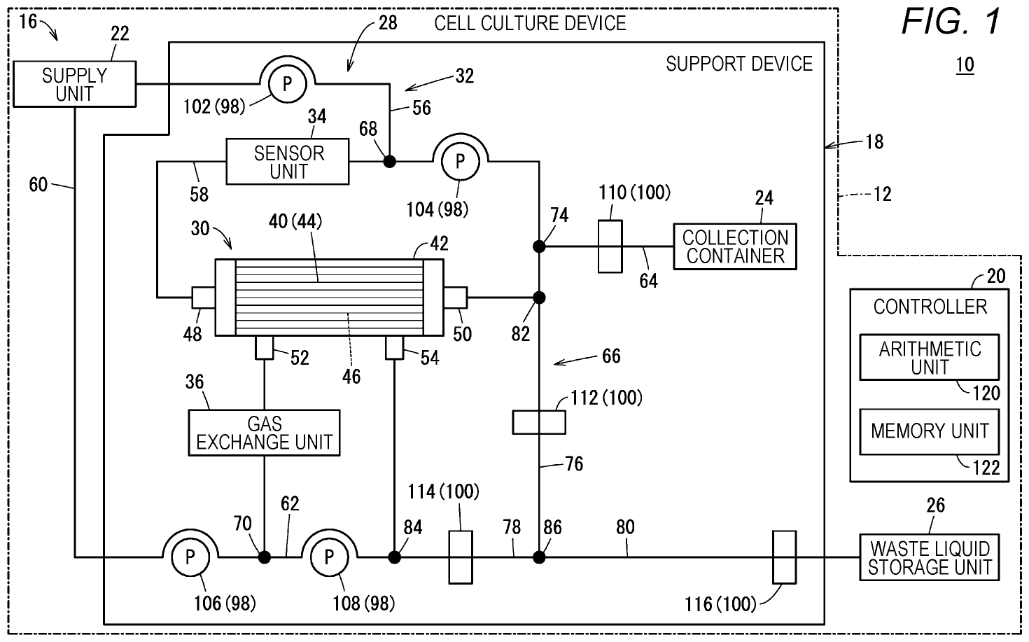

FIG. 1 is a schematic of an example cell culture device 10 for culturing cells. The cell culture device 10 may include a calibration device 12 and a sensor unit 34. The calibration device 12 may be configured to calibrate measurement values of the sensor unit 34.

The cell culture device 10 may be configured to culture cells separated from biological tissue. The cells may be included in a culture medium that is fed into the cell culture device 10. The cells may include, for example, adherent cells or floating cells. In at least one example embodiment, the cells may include embryonic stem (ES) cells, induced pluripotent stem (iPS) cells, mesenchymal stem cells, or any combination thereof.

The cell culture device 10 may include a cell culture circuit 16, a support device 18, and a controller 20. The cell culture circuit 16 may be configured to support the flow of a liquid. The liquid may include, for example, a cell fluid, a culture medium, a washing liquid, a stripping liquid, or any combination thereof.

The cell fluid is a solution including cells. The culture medium is a culture solution for growing cells. The culture medium may be selected according to the cell to be cultured. In at least one example embodiment, the culture medium may include minimum essential media (MEM). The washing liquid may be used to wash the inside of the cell culture circuit 16. The washing liquid may include, for example, water, a buffer solution, physiological saline, the like, or any combination thereof. In at least one example embodiment, the buffer solution may include phosphate buffered salts (PBS), tris-buffered saline (TBS), the like, or any combination thereof. The stripping liquid may be used to detach cells from a bioreactor 30 of the cell culture circuit 16. In at least one example embodiment, the stripping liquid may include trypsin, an ethylenediaminetetraacetic acid (EDTA) solution, the like, or any combination thereof.

The cell culture circuit 16 may be a disposable product. For example, in at least one example embodiment, the cell culture circuit 16 may be discarded after a single use. In at least one example embodiment, the cell culture circuit 16 may be discarded every time a predetermined number of cells are cultured.

The cell culture circuit 16 may include a supply unit 22, a collection container 24, a waste liquid storage unit 26, and a culture body 28. The supply unit 22 may supply one or more of the cell fluid, the culture medium, the washing liquid, and the stripping liquid to the culture body 28. Each of the liquids may be provided in a medical bag or container. The collection container 24 may collect the cells cultured in the culture body 28. In at least one example embodiment, the collection container 24 may include a medical bag or container. In at least one example embodiment, the collection container 24 may include a tank or other container prepared from a hard material. The waste liquid storage unit 26 may store the waste liquid generated in the culture body 28. In at least one example embodiment, the waste liquid storage unit 26 may include a medical bag or container. In at least one example embodiment, the waste liquid storage unit 26 may include a tank or other container prepared from a hard material.

The culture body 28 may include a bioreactor 30, a flow path 32, a sensor unit 34, and a gas exchange unit 36. The bioreactor 30 may include a plurality of hollow fiber membranes 40 and a housing 42 surrounding or supporting the plurality of hollow fiber membranes 40. The housing 42 may have a first end portion and an opposing second end portion. In at least one example embodiment, the housing 42 may be a cylindrical housing. In at least one example embodiment, one end portion of each hollow fiber membrane of the plurality of hollow fiber membranes 40 may be fixed to the first end portion of the housing 42, and the other ends of each hollow fiber membrane of the plurality of hollow fiber membranes 40 may be fixed to the second end portion of the housing 42. Each hollow fiber membrane of the plurality of hollow fiber membranes 40 may include a polymer material.

The bioreactor 30 may include a first region 44 and a second region 46. The first region 44 may be defined by inner holes of each hollow fiber membrane of the plurality of hollow fiber membranes 40. The second region 46 may be defined by a space between the inner peripheral surface of the housing 42 and the outer peripheral surfaces of the plurality of hollow fiber membranes 40. Each hollow fiber membrane of the plurality of hollow fiber membranes 40 may have a plurality of pores. The first region 44 and the second region 46 may communicate with each other through the plurality of pores. The diameter of each pore may be a size that allows passage of small molecules (such as, for example, water, ions, oxygen, lactate, and the like) while blocking passage of macromolecules (such as, for example, cells and the like). In at least one example embodiment, the diameter of each pore may be greater than or equal to about 0.005 micrometers to less than or equal to about 10 micrometers.

A first inlet port 48, a first outlet port 50, a second inlet port 52, and a second outlet port 54 may be attached to the housing 42. The first inlet port 48 may be attached to one end (e.g., the first end portion) of the housing 42. The first inlet port 48 may communicate with the first region 44 via an inlet located at one end of the plurality of hollow fiber membranes 40. The first outlet port 50 may be attached to the other end (e.g., the second end portion) of the housing 42. The first outlet port 50 may communicate with the first region 44 via an outlet located at the other end of the plurality of hollow fiber membranes 40.

The second inlet port 52 and the second outlet port 54 may be attached to the outer peripheral surface of the housing 42. The second inlet port 52 may be located between the center of the housing 42 and the first inlet port 48 in the longitudinal direction of the housing 42. The second outlet port 54 may be located between the center of the housing 42 and the first outlet port 50 in the longitudinal direction of the housing 42. The second inlet port 52 and the second outlet port 54 may both communicate with the second region 46.

The flow path 32 may include a plurality of tubes through which a liquid flows. In at least one example embodiment, each tube may be prepared from a soft resin material. The flow path 32 may include a first supply flow path 56, a first circulation flow path 58, a second supply flow path 60, a second circulation flow path 62, a collection flow path 64, and a waste liquid flow path 66. One end (e.g., a first end) of the first supply flow path 56 may by connected to the supply unit 22. The other end (e.g., a second end) of the first supply flow path 56 may be connected to a first junction portion 68 of the first circulation flow path 58.

The first junction portion 68 may be located at an intermediate part of the first circulation flow path 58 in the extending direction. One end of the first circulation flow path 58 may be connected to the first inlet port 48. The other end of the first circulation flow path 58 may be connected to the first outlet port 50. The first circulation flow path 58 may communicate with the first region 44.

One end (e.g., a first end) of the second supply flow path 60 may be connected to the supply unit 22. The supply unit 22 may supply the culture medium to the second supply flow path 60 at a first predetermined time. The supply unit 22 may supply the washing liquid to the second supply flow path 60 at a second predetermined time. The other end (e.g., a second end) of the second supply flow path 60 may be connected to a second junction portion 70 of the second circulation flow path 62.

The second junction portion 70 may be located at an intermediate part of the second circulation flow path 62 in the extending direction. One end (e.g., a first end) of the second circulation flow path 62 may be connected to the second inlet port 52. The other end (e.g., a second end) of the second circulation flow path 62 may be connected to the second outlet port 54. The second circulation flow path 62 may communicate with the second region 46.

The supply unit 22 may individually supply the cell fluid, the culture medium, the washing liquid, and the stripping liquid to the first circulation flow path 58 via the first supply flow path 56 at predetermined times. The supply unit 22 may supply the culture medium to the second circulation flow path 62 via the second supply flow path 60 at a predetermined time.

The collection flow path 64 may extend from the first circulation flow path 58. One end of the collection flow path 64 may be connected to a collection branch portion 74 of the first circulation flow path 58. The collection branch portion 74 may be positioned between the first junction portion 68 and the first outlet port 50 in the first circulation flow path 58. The other end of the collection flow path 64 may be connected to the collection container 24.

A liquid to be discarded may flow through the waste liquid flow path 66. The liquid to be discarded may flow from the first circulation flow path 58, the second circulation flow path 62, or a combination thereof. The waste liquid flow path 66 may include a first waste liquid flow path 76, a second waste liquid flow path 78, and a third waste liquid flow path 80. The first waste liquid flow path 76 may extend from the first circulation flow path 58. One end (e.g., a first end) of the first waste liquid flow path 76 may be connected to a first branch portion 82 of the first circulation flow path 58. The first branch portion 82 may be positioned between the first outlet port 50 of the first circulation flow path 58 and the collection branch portion 74. The second waste liquid flow path 78 may extend from the second circulation flow path 62. One end (e.g., a first end) of the second waste liquid flow path 78 may be connected to the second branch portion 84 of the second circulation flow path 62. The second branch portion 84 may be located between the second junction portion 70 of the second circulation flow path 62 and the second outlet port 54. The other end (e.g., a second end) of the first waste liquid flow path 76 and the other end (e.g., a second end) of the second waste liquid flow path 78 may be connected to each other at an intermediate junction portion 86. One end (e.g., a first end) of the third waste liquid flow path 80 may be connected to the first waste liquid flow path 76 and the second waste liquid flow path 78 at the intermediate junction portion 86. The other end (e.g., a second end) of the third waste liquid flow path 80 may be connected to the waste liquid storage unit 26.

The sensor unit 34 may be provided between the first junction portion 68 of the first circulation flow path 58 and the first inlet port 48. The sensor unit 34 may include a sensor 130 for turbidity measurement. The relationship between the measurement value of the sensor 130 and the true value of the turbidity may be preset. A turbidity measurement value as measured by the sensor 130 may be converted into the cell concentration by a calibration unit 126.

The gas exchange unit 36 may be attached between the second junction portion 70 of the second circulation flow path 62 and the second inlet port 52. The gas exchange unit 36 may allow certain gas or gases to pass through a liquid (e.g., culture medium) flowing through the second circulation flow path 62. The gas or gases used in the gas exchange unit 36 may include those found naturally in air. For example, the gas moving through the gas exchange unit 36 may include nitrogen, oxygen, carbon dioxide, or any combination thereof. In at least one example embodiment, the gas moving through the gas exchange unit 36 may include about 75% of nitrogen, about 20% of oxygen, and about 5% of carbon dioxide in volume ratio.

The cell culture circuit 16 may be set within the support device 18. The support device 18 may include a cassette that supports the cell culture circuit 16. The support device 18 may be a reusable product that can be used several times.

The support device 18 may include a plurality of pumps 98 and a plurality of clamps 100. Each of the plurality of pumps 98 may be configured to apply a flow force to the liquid in the flow path 32, for example, by squeezing the wall of the flow path 32. Each of the plurality of pumps 98 may include a pressing member. The pressing member may include, for example, a rotating member and a plurality of pressing rollers. The plurality of pressing rollers may be attached to an outer peripheral portion of the rotating member. The plurality of pressing rollers may be arranged at intervals in the circumferential direction of the rotating member. Each pressing roller may rub the outer surface of the wall portion of the flow path 32.

The plurality of pumps 98 may include a first supply pump 102, a first circulation pump 104, a second supply pump 106, and a second circulation pump 108. A state in which the cell culture circuit 16 is set in the support device 18, for example, as illustrated in FIG. 1, may be simply referred to as a “set state”.

In the set state, a part of the first supply flow path 56 may be attached to the first supply pump 102. The first supply pump 102 may apply a flow force in a direction from the supply unit 22 toward the first circulation flow path 58 to the liquid in the first supply flow path 56.

In the set state, a part of first circulation flow path 58 may be attached to first circulation pump 104. The first circulation pump 104 may apply a flow force in a direction from the first outlet port 50 toward the first inlet port 48 to the liquid in the first circulation flow path 58. The first circulation pump 104 may also apply a flow force in a direction from the first inlet port 48 toward the first outlet port 50 to the liquid in the first circulation flow path 58.

In the set state, a part of the second supply flow path 60 may be attached to the second supply pump 106. The second supply pump 106 may apply a flow force in a direction from the supply unit 22 toward the second circulation flow path 62 to the liquid in the second supply flow path 60.

In the set state, a part of the second circulation flow path 62 may be attached to the second circulation pump 108. The second circulation pump 108 may apply a flow force in a direction from the second outlet port 54 toward the second inlet port 52 to the liquid in the second circulation flow path 62. The second circulation pump 108 may also apply a flow force in a direction from the second inlet port 52 toward the second outlet port 54 to the liquid in the second circulation flow path 62.

The plurality of clamps 100 may be configured to close the flow path 32 by pressing the outer surface of the flow path 32 toward the inner surface. For example, the plurality of clamps 100 may be on-off valves. The plurality of clamps 100 may include a collection clamp 110, a first waste liquid clamp 112, a second waste liquid clamp 114, and a third waste liquid clamp 116.

In the set state, a part of the collection flow path 64 may be attached to the collection clamp 110. The collection clamp 110 may open and close the collection flow path 64. In the set state, a part of the first waste liquid flow path 76 may be attached to the first waste liquid clamp 112. The first waste liquid clamp 112 may open and close the first waste liquid flow path 76. In the set state, a part of the second waste liquid flow path 78 may be attached to the second waste liquid clamp 114. The second waste liquid clamp 114 may open and close the second waste liquid flow path 78. In the set state, a part of the third waste liquid flow path 80 may be attached to the third waste liquid clamp 116. The third waste liquid clamp 116 may open and close the third waste liquid flow path 80.

The controller 20 may be, for example, a computer. The controller 20 may include an arithmetic unit 120, a memory unit 122, and various drive circuits.

The arithmetic unit 120 may include a processing circuit. The processing circuit may include a processor, such as a CPU. The processing circuit may be an integrated circuit such as an ASIC or an FPGA. The processor may be configured to execute various types of processing, for example, by executing a program stored in the memory unit 122. At least some of the plurality of processing may be performed by an electronic circuit including a discrete device.

The memory unit 122 may include a volatile memory and a nonvolatile memory. The volatile memory may include a RAM, the like, or a combination thereof. The volatile memory may be used as a working memory of the processor. The volatile memory may be configured to temporarily store data and the like necessary for processing or computation. Examples of the nonvolatile memory may include a ROM and a flash memory. The non-volatile memory may be used as a storage memory. The nonvolatile memory may store programs, tables, maps, the like, or any combination thereof. At least a part of the memory unit 122 may be provided in the processor, the integrated circuit, the like, or any combination thereof.

The calibration device 12 may use, or include, the supply unit 22, the first supply flow path 56, the first supply pump 102, the first circulation flow path 58, the first circulation pump 104, the bioreactor 30, the sensor unit 34, and the controller 20.

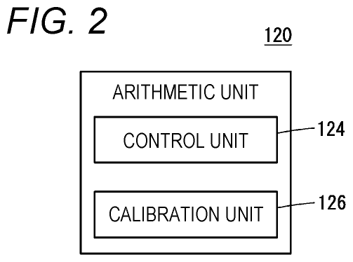

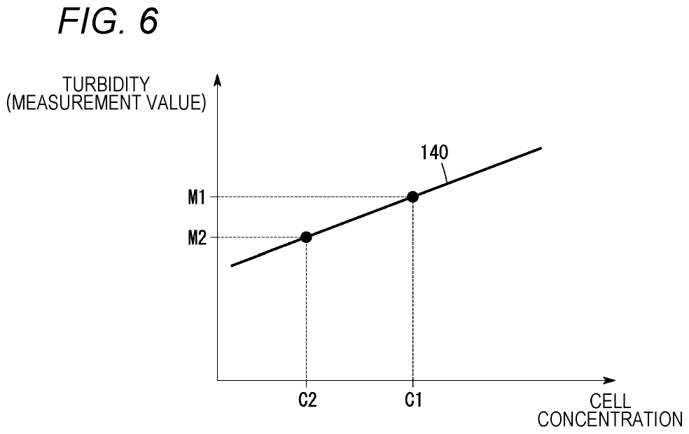

FIG. 2 is a block diagram illustrating the arithmetic unit 120. The arithmetic unit 120 of the controller 20 may function as the control unit 124 and the calibration unit 126. The control unit 124 may control the first supply pump 102, the first circulation pump 104, valves 134, or any combination thereof. The calibration unit 126 may acquire the measurement value of the turbidity from the sensor 130 and may create a calibration curve 140 (see FIG. 6) between the measurement value of the turbidity and the cell concentration.

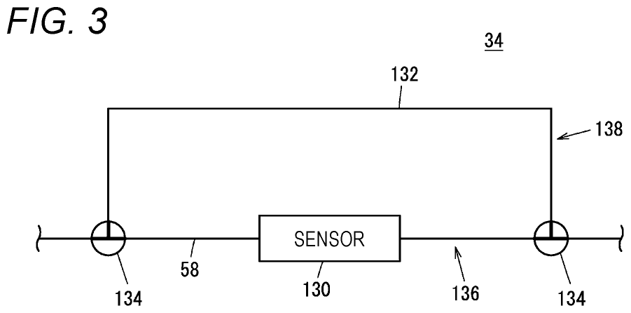

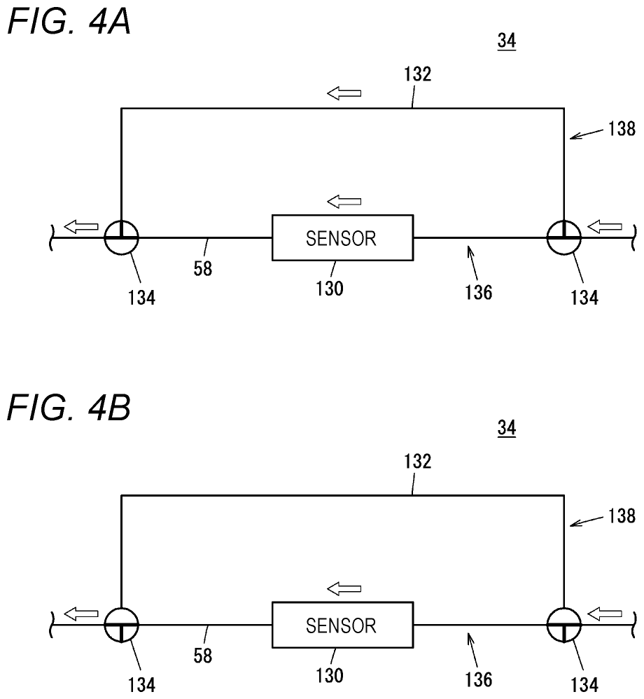

FIG. 3 is a schematic of an example configuration of the sensor unit 34. FIGS. 4A and 4B are schematics illustrating connection states between the first circulation flow path 58 and the dilution flow path 132 in the sensor unit 34 as illustrated in FIG. 3. The sensor unit 34 may include the first circulation flow path 58, the sensor 130, the dilution flow path 132, and the two valves 134.

In at least one example embodiment, the sensor 130 may be a turbidity sensor. The sensor 130 may be connected to the first circulation flow path 58 in series. The dilution flow path 132 may be a tube member. The dilution flow path 132 may be connected to sensor 130 of first circulation flow path 58 in parallel. The volume of the dilution flow path 132, the volume of the first circulation flow path 58, and the volume of the bioreactor 30 may be stored in the memory unit 122 in advance. For example, the volume of the dilution flow path 132 may be equal to the sum of the volume of the first circulation flow path 58 and the volume of the bioreactor 30. The valve 134 may be disposed at each connection point between the first circulation flow path 58 and the dilution flow path 132.

The two valves 134 may include three-way valves. As illustrated in FIG. 4A, the two valves 134 may allow for communication between the first circulation flow path 58 and the dilution flow path 132. As illustrated in FIG. 4B, the two valves 134 may also block or stop communication between the first circulation flow path 58 and the dilution flow path 132. The two valves 134 may operate according to a command signal output from the control unit 124.

The first circuit 136 may be formed by a closed circuit. The close circuit may include, for example, the first circulation flow path 58, the sensor 130, the bioreactor 30, and the first circulation pump 104. The second circuit 138 may be formed by the dilution flow path 132 connected to the first circuit 136 in parallel.

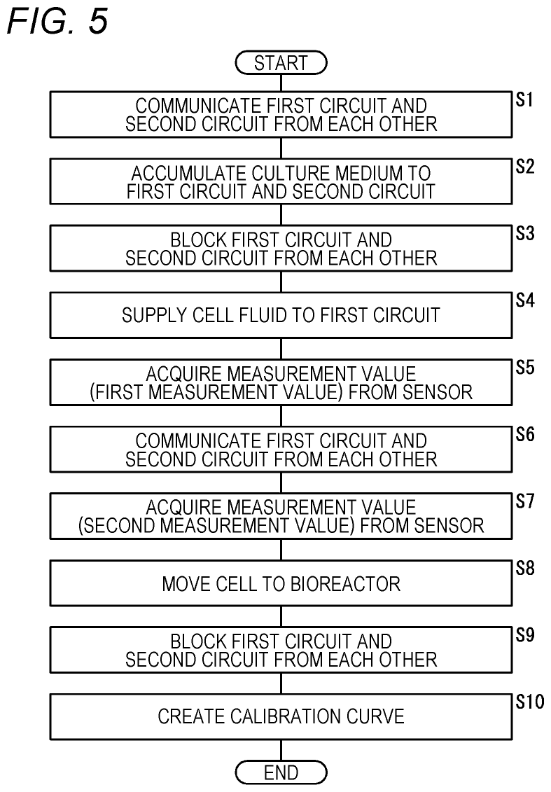

FIG. 5 is a flowchart of an example calibration method for the cell culture device 10. Before the calibration process, such as illustrated in FIG. 5, a user or operator of the cell culture device 10 receives, or counts, the number of cells in the cell fluid, for example, from or using a cell counter. The user or operator may fill a medical bag or container with the cell fluid for which the number of cells has been counted. The user or operator may set the medical bag or container including the cell fluid for which the number of cells has been counted or known in the supply unit 22. The user or operator may fill the medical bag or container with a culture medium that does not include cells. The user or operator may set the medical bag or container including the culture medium in the supply unit 22. The user or operator may enter the counted or known number of cells into the memory unit 122 of the cell culture device 10.

The method may include, in step S1, causing, for example, using the control unit 124, the dilution flow path 132 and the first circulation flow path 58 to communicate with each other. In at least one example embodiment, the causing may include alternating, or changing, the state of the valve 134.

The method may include, in step S2, connecting (or causing to be connected), for example, using the control unit 124, the first supply flow path 56 to the medical bag or container of the culture medium. The method may include, in step S2, operating, for example, using the control unit 124, the first supply pump 102 and the first circulation pump 104. According to the operations of the first supply pump 102 and the first circulation pump 104, the supply unit 22 may supply the culture medium, which includes no cells, to the first circulation flow path 58. As a result, the culture medium, which does not include any cells, may accumulate in both the first circuit 136 and the second circuit 138. The method may include, in step S2, after supplying the culture medium, stopping, for example, using the control unit 124, the first supply pump 102.

The method may include, in step S3, blocking (or causing to be blocked), for example, using the control unit 124, the dilution flow path 132 and the first circulation flow path 58 from each other. In at least one example embodiment, the blocking may include alternating, or changing, the state of the valve 134 (FIG. 4B).

The method may include, in step S4, connecting (or causing to be connected), for example, using the control unit 124, the first supply flow path 56 to the medical bag or container including the cell fluid. The method may include, in step S4, operating, for example, using the control unit 124, the first supply pump 102 and the first circulation pump 104. The supply unit 22 may supply the cell fluid to the first circulation flow path 58 according to the operations of the first supply pump 102 and the first circulation pump 104. The method may include, in step S4, blocking (or causing to be block), for example, using the control unit 124, the dilution flow path 132 from the first circulation flow path 58, such that, in the sensor unit 34, the cell fluid is supplied only to the first circuit 136. The method may include, in step S4, circulating (or causing to be circulated), for example, using the control unit 124, the cell fluid for a predetermined time after the supplying of the cell fluid is completed. The method may include, in step S4, stopping (or causing to be stopped), for example, using the control unit 124, the first supply pump 102. The first supply pump 102 may be stopped after the cell fluid is uniform and stabilized.

The method may include, in step S5, acquiring, for example, using the calibration unit 126, the measurement value from the sensor 130. This measurement value may be referred to as a first measurement value M1. The memory unit 122 may store the first measurement value M1.

The method may include, in step S6, causing, for example, using the control unit 124, the dilution flow path 132 and the first circulation flow path 58 to communicate with each other. In at least one example embodiment the causing may include alternating, or changing, the state of the valve 134. The method may include, in step S6, mixing (or causing to be mixed), for example, using the control unit 124, the culture medium of the second circuit 138 and the culture medium of the first circuit 136 such that the culture medium containing the cells is diluted.

The method may include, in step S7, acquiring, for example, by the calibration unit 126, the measurement value from the sensor 130. This measurement value may be referred to as a second measurement value M2. The memory unit 122 may store the second measurement value M2.

The method may include, in step S8, moving (or causing to be moved), for example, using the control unit 124, the cells in the sensor unit 34 to the bioreactor 30. The method may include, in step S8, connecting (or causing to be connected), for example, using, the control unit 124, the first supply flow path 56 and the medical bag of the culture medium. The method may include, in step S8, operating, for example, using the control unit 124, the first supply pump 102 and the first circulation pump 104. According to the operations of the first supply pump 102 and the first circulation pump 104, the supply unit 22 may be caused to supply the culture medium containing no cells to the first circulation flow path 58. In this manner, the method may include, in step S8, causing, the cells in the sensor unit 34 to flow to the bioreactor 30.

The method may include, in step S9, blocking (or causing to be blocked), for example, using the control unit 124, the dilution flow path 132 and the first circulation flow path 58 from each other. In at least one example embodiment, the blocking may include alternating, or changing, the state of the valve 134.

The method may include, in step S10, creating (or causing to be created), for example, using the calibration unit 126, the calibration curve 140. The method may include, at step S10, computing (or causing to be computed), for example, using the calibration unit 126, the cell concentration of the culture medium in the first circuit 136 at the time of step S5. The method may include, at step S10, computing (or causing to be computed), for example, using calibration unit 126, the cell concentration based on the information (e.g., volume and number of cells of the first circuit 136) stored in the memory unit 122. This cell concentration may be defined as a first concentration C1. The method may include, in step S10, associating (or causing to be associated), for example, using the calibration unit 126, the first concentration C1 with the first measurement value M1. The method may include, at step S10, computing (or causing to be computed), for example, using the calibration unit 126, the cell concentrations of the culture media of the first circuit 136 and the second circuit 138 at the time of step S7. The method may include, at step S10, computing (or causing to be computed), for example, using calibration unit 126, the cell concentration based on the information (e.g., volume of the first circuit 136, the volume of the second circuit 138, and number of cells of circuits) stored in the memory unit 122. This cell concentration may be defined as a second concentration C2. The method may include, at step S10, associating (or causing to be associated), for example, using the calibration unit 126 the second concentration C2 with the second measurement value M2. The cell concentration and the turbidity may have a linear relationship. The method may include, at step S10, creating, for example, using the calibration unit 126 the calibration curve 140 of the cell concentration and the turbidity (measurement value) from the first concentration C1, the first measurement value M1, the second concentration C2, and the second measurement value M2. The method may include, at step S10, storing (or causing to be stored), for example, using the calibration unit 126, the calibration curve 140 in the memory unit 122. In at least one example embodiment, the first concentration C1 may be computed at the time of step S5, that is, when the memory unit 122 stores the first measurement value M1. In at least one example embodiment, the second concentration C2 may be calculated at the time of step S7, that is, at the time when the memory unit 122 stores the second measurement value M2.

In at least one example embodiment, one type of cell fluid may be supplied to the cell culture circuit 16, and the cell fluid may be diluted with a dilution culture medium provided in advance in the cell culture circuit 16. In such instances, it is not necessary to prepare a plurality of concentrations of cell fluid. It is also not necessary to discard the cell fluid and the culture medium, which helps to reduce, or eliminate, waste. Further still, the calibration work is performed by, or using, the cell culture device 10, which helps to improve efficiency and automation.

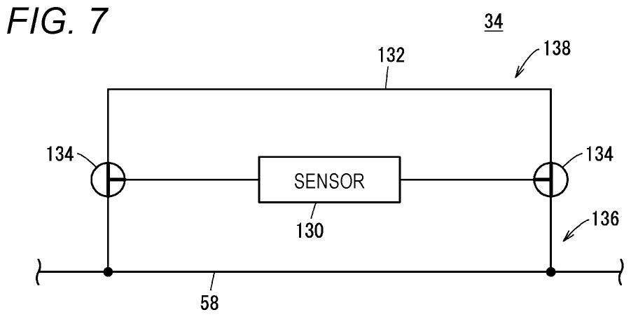

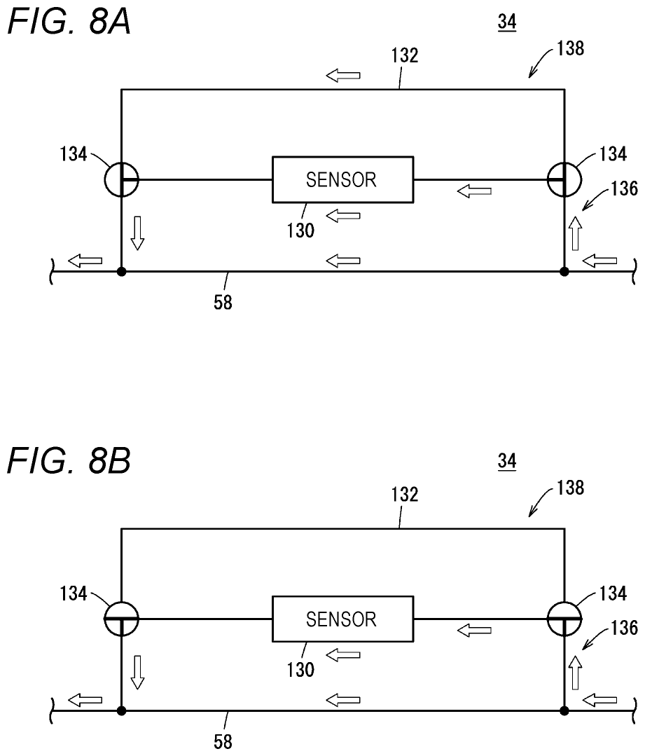

FIG. 7 is a schematic of another example configuration of the sensor unit 34. FIGS. 8A and 8B are schematics illustrating a connection state between the first circulation flow path 58 and the dilution flow path 132 in the sensor unit 34 as illustrated in FIG. 7.

In the sensor unit 34 as illustrated in FIG. 3, the sensor 130 may be connected to the first circulation flow path 58 in series. On the other hand, in the sensor unit 34 as illustrated in FIG. 7, the sensor 130 may be connected to the first circulation flow path 58 in parallel. The sensor unit 34 as illustrated in FIG. 7 is substantially the same as the sensor unit 34 as illustrated in FIG. 3 except that the sensors 130 are connected in parallel in the instance of FIG. 7 and in series in the instance of FIG. 3.

As illustrated in FIG. 7, in the instance of parallel alignment, the first circuit 136 may be formed by a closed circuit including the first circulation flow path 58, the sensor 130, the bioreactor 30, and the first circulation pump 104. Furthermore, as illustrated in FIG. 7, in the instance of parallel alignment, the second circuit 138 may be formed by the dilution flow path 132 connected to the first circuit 136 in parallel.

The internal flow path of the sensor 130 is comparatively thin, such that when the sensor 130 is connected to the first circulation flow path 58 in series as illustrated in FIG. 3, the flow rate of the culture medium flowing through the first circulation flow path 58 may be affected by an increase in internal pressure in the internal flow path and the circuit of the sensor 130. On the other hand, when the sensor 130 is connected to the first circulation flow path 58 in parallel as illustrated in FIG. 7, the flow rate of the culture medium flowing through the first circulation flow path 58 may not be affected by the increase in the internal pressure in the internal flow path of the sensor 130 and the circuit. Accordingly, a comparatively large volume of the cell fluid and the culture medium can flow into the first circulation flow path 58, while also achieving the same effects as in the instance of the series alignment.

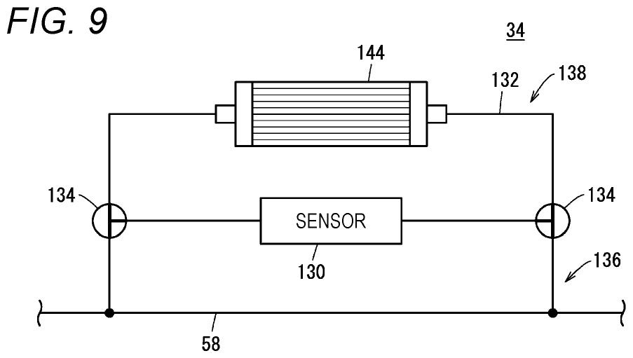

FIG. 9 is a schematic of another example configuration of the sensor unit 34.

As illustrated in FIG. 9, a sub-bioreactor 144 may be provided in the dilution flow path 132. The sub-bioreactor 144 may be connected to the dilution flow path 132 in series. The sub-bioreactor 144 may be of the same construction as the bioreactor 30. In FIG. 9, another circulation flow path (for example, corresponding to the second circulation flow path 62 of the bioreactor 30) connected to the sub-bioreactor 144 is omitted.

As illustrated in FIG. 9, in this instance, the first circuit 136 may be formed by a closed circuit including the first circulation flow path 58, the sensor 130, the bioreactor 30, and the first circulation pump 104. Furthermore, in the instance of FIG. 9, the second circuit 138 may be formed by the dilution flow path 132 connected to the first circuit 136 in parallel and the sub-bioreactor 144.

In the instance of FIG. 9, the cells may be cultured by both the bioreactor 30 and the sub-bioreactor 144, which helps to increase the number of cells that can be cultured, while also achieving the same effects as in the instance of sensor unit 34 as illustrated in FIG. 3.

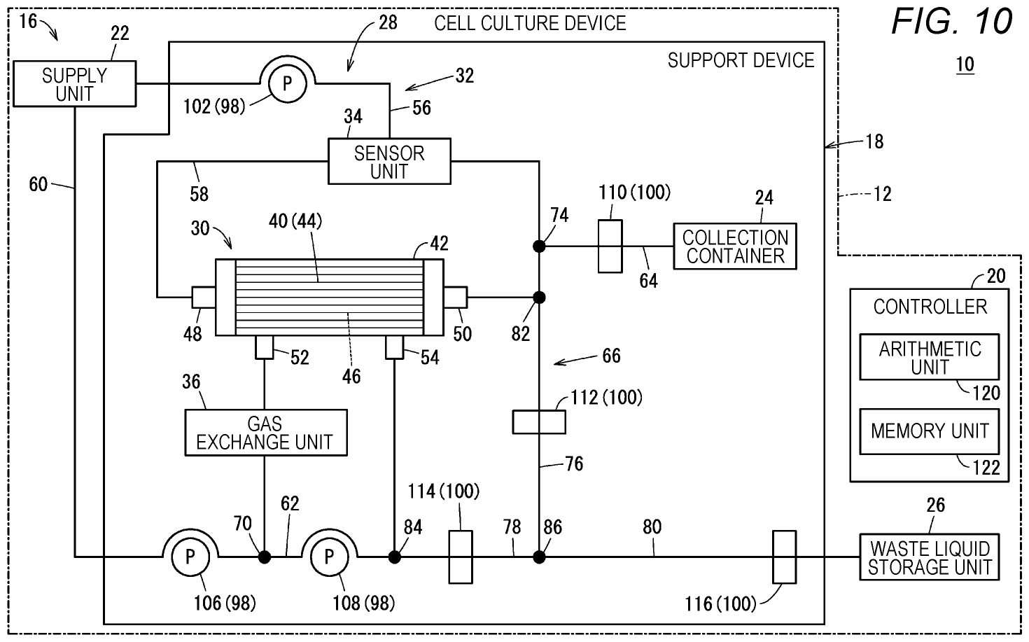

FIG. 10 is a schematic of another example configuration of the cell culture device 10. The cell culture device 10 as illustrated in FIG. 10 differs from the cell culture device 10 as illustrated in FIG. 1 in that the positioning of the sensor unit 34. As illustrated in FIG. 10, the sensor unit 34 may be provided between the collection branch portion 74 of the first circulation flow path 58 and the first inlet port 48.

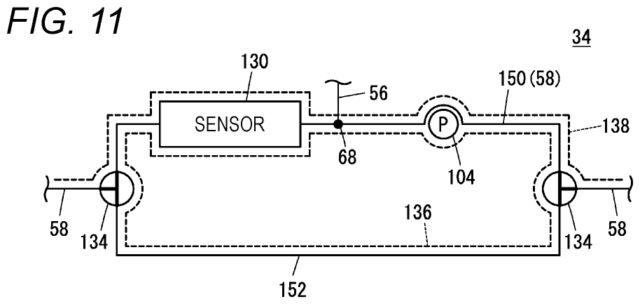

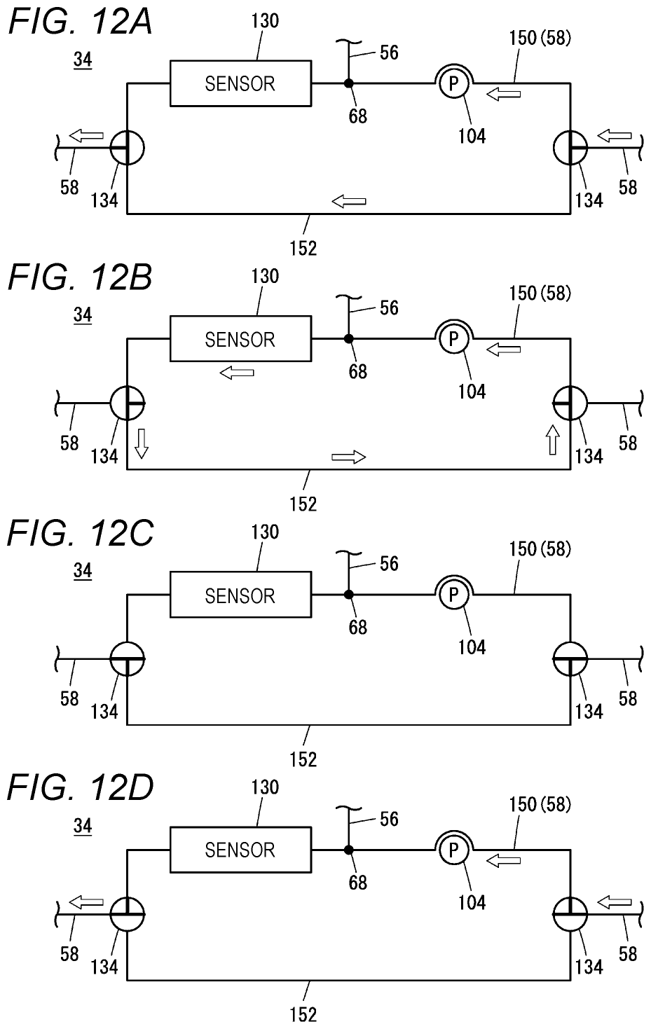

FIG. 11 is a schematic of another example configuration of the sensor unit 34. FIGS. 12A-12D are schematics illustrating a connection state of the first circulation flow path 58, the sensor flow path 150, and the concentration flow path 152 in the sensor unit 34 as illustrated in FIG. 11. The sensor unit 34 may include the sensor flow path 150, the sensor 130, the concentration flow path 152, and the two valves 134.

As illustrated in FIG. 11, the sensor flow path 150 may be a part of the first circulation flow path 58. The sensor flow path 150 may be a portion of the first circulation flow path 58 sandwiched between the two valves 134. The sensor flow path 150 may include the first junction portion 68. Furthermore, the sensor flow path 150 may be attached to the first circulation pump 104. The sensor 130 may be connected to the sensor flow path 150 in series. The concentration flow path 152 may include a tube member. The concentration flow path 152 may be connected to the sensor flow path 150 of the first circulation flow path 58 in parallel. The volume of the first circulation flow path 58, the volume of the sensor flow path 150, the volume of the concentration flow path 152, and the volume of the bioreactor 30 may be stored in the memory unit 122 in advance. The valve 134 may be disposed at each connection point between the sensor flow path 150 and the concentration flow path 152.

The two valves 134 may each include, for example, three-way valves. As illustrated in FIGS. 12A to 12D, each of the two valves 134 may be switch between the communicating state and the block state between the sensor flow path 150 and the concentration flow path 152. Each of the two valves 134 may be switched between a communicating state and a block state between a portion of the first circulation flow path 58 other than the sensor flow path 150 and the sensor flow path 150 (and the concentration flow path 152).

As illustrated in FIG. 11, the first circuit 136 may be formed by a closed circuit including the sensor flow path 150, the concentration flow path 152, the sensor 130, and the first circulation pump 104. The second circuit 138 may be formed by a closed circuit including the first circulation flow path 58 (including the sensor flow path 150), the sensor 130, and the bioreactor 30.

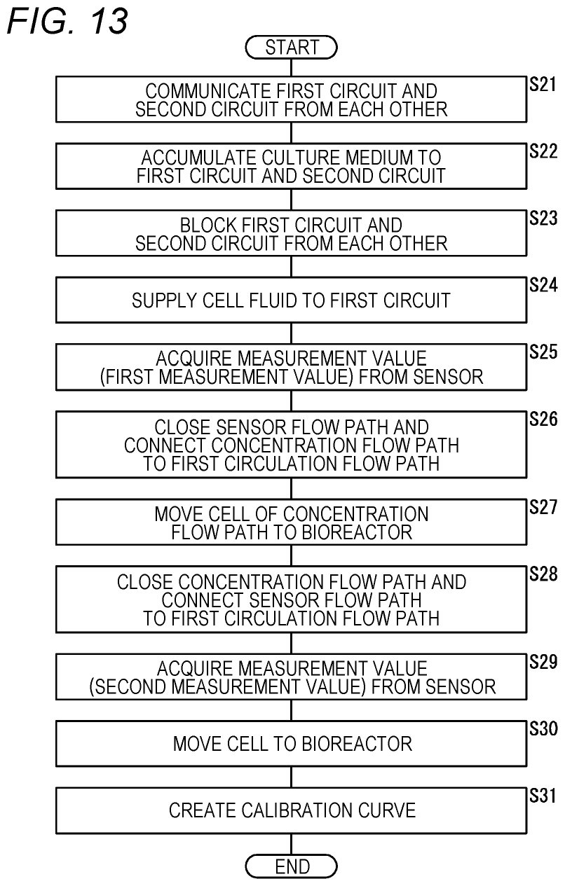

FIG. 13 is a flowchart illustrating another example calibration method. Before the calibration process, such as illustrated in FIG. 13, a user or operator of the cell culture device 10 receives, or counts, the number of cells in the cell fluid, for example, from or using a cell counter. The user or operator may fill a medical bag or container with the cell fluid for which the number of cells has been counted. The user or operator may set the medical bag or container including the cell fluid for which the number of cells has been counted, or is known, in the supply unit 22. the user or operator may fill the medical bag or container with a culture medium that does not include cells. The user or operator may set the medical bag or container including the culture medium in the supply unit 22. The user or operator may enter the counted or known number of cells into the memory unit 122 of the cell culture device 10. The user or operator may fill the culture medium in the medical bag or container. The user or operator may set the medical bag or the container in the supply unit 22.

The method may include, in step S21, controlling, for example, using the control unit 124, the valve 134 to cause a portion of the first circulation flow path 58 other than the sensor flow path 150 and the sensor flow path 150 to communicate with each other. The method may include, in step S21, causing, for example, using the control unit 124, the first circulation flow path 58 and the concentration flow path 152 to communicate with each other.

The method may include, in step S22, connecting (or causing to be connected), for example, using the control unit 124, the first supply flow path 56 to the medical bag of the culture medium. The method may include, in step S22, operating, for example, using the control unit 124, the first supply pump 102 and the first circulation pump 104. According to the operations of the first supply pump 102 and the first circulation pump 104, the supply unit 22 may supply the culture medium that includes no cells to the first circulation flow path 58. As a result, the culture medium including no cells may accumulate in both the first circuit 136 and the second circuit 138. The method may include, in step S22, stopping, for example, using the control unit 124, the first supply pump 102.

The method may include, in step S23, blocking (or causing to be blocked), for example, using the control unit 124, a portion of the first circulation flow path 58 other than the sensor flow path 150 from the sensor flow path 150. In at least one example embodiment, the blocking may include alternating, or changing, the state of the of the valve 134. The method may include, in step S23, blocking, for example, using the control unit 124, the first circulation flow path 58 and the concentration flow path 152 from each other. The method may include, in step S23, causing, for example, using the control unit 124, the sensor flow path 150 and the concentration flow path 152 to communicate with each other. In this manner, in Step S23, the first circuit 136 may be formed. Furthermore, the first circuit 136 and the second circuit 138 may be cut off from each other.

The method may include, in step S24, connecting (or causing to be connected), for example, using the control unit 124, the first supply flow path 56 to the medical bag or container of the cell fluid. The method may include, in step S24, operating, for example, using the control unit 124, the first supply pump 102 and the first circulation pump 104. The supply unit 22 may supply the cell fluid to the sensor flow path 150 in accordance with the operations of the first supply pump 102 and the first circulation pump 104. The method may include, in step S24, blocking (or causing to be blocked), for example, using the control unit 124, the first circulation flow path 58 other than the sensor flow path 150 from the sensor flow path 150. In this manner, in the sensor unit 34, the cell fluid may be supplied only to the first circuit 136. The method may include, in step S24, stopping, for example, using the control unit 124, the first supply pump 102 after the supply of the cell fluid is completed.

The method may include, in step S25, acquiring, for example, using the calibration unit 126, the measurement value from the sensor 130. This measurement value may be referred to as a first measurement value M1. The memory unit 122 may store the first measurement value M1.

The method may include, in step S26, causing, for example, using the control unit 124, the concentration flow path 152 and the first circulation flow path 58 to communicate with each other. In at least one example embodiment, communication between the concentration flow path 152 and the first circulation flow path 58 may be controlled by the valve 134. In at least one example embodiment, the causing may include alternating, or changing, the state of the valve 134. The method may include, in step S26, blocking, for example, using the control unit 124, a portion of the first circulation flow path 58 other than the sensor flow path 150 from the sensor flow path 150. The method may include, in step S26, blocking (or causing to be blocked), for example, using the control unit 124, the sensor flow path 150 and the concentration flow path 152 from each other.

The method may include, in step S27, moving (or causing to move), for example, using the control unit 124, the cells in the sensor unit 34 to the bioreactor 30. As in instance of step S8 as illustrated in FIG. 5, the method as illustrated in FIG. 13, may include, in step S27, operating, for example, using the control unit 124, the first supply pump 102 and the first circulation pump 104 to supply the culture medium that does not include cells from the supply unit 22 to the first circulation flow path 58.

The method may include, in step S28, controlling, for example, using the control unit 124, the valve 134 to cause a portion of the first circulation flow path 58 other than the sensor flow path 150 and the sensor flow path 150 to communicate with each other. The method may include, in step S28, blocking (or causing to be blocked), for example, using the control unit 124, the first circulation flow path 58 and the concentration flow path 152 from each other. The method may include, in step S28, blocking (or causing to be blocked), for example, using the control unit 124, the sensor flow path 150 and the concentration flow path 152 from each other. In this manner, the second circuit 138 may be formed. The method may include, in step S28, mixing (or causing to be mixed), for example, using the control unit 124, the culture medium other than the sensor flow path 150 in the first circulation flow path 58 and the culture medium such that the culture medium containing the cells is diluted.

The method may include, in step S29, acquiring, for example, using the calibration unit 126, the measurement value from the sensor 130. This measurement value may be referred to as a second measurement value M2. The memory unit 122 may store the second measurement value M2.

The method may include, in step S30, moving (or causing to move), for example, using the control unit 124, the cells contained in the culture medium to the bioreactor 30.

The method may include, in step S31, creating (or causing to be created), for example, using the calibration unit 126, the calibration curve 140. The method may include, at step S31, computing (or causing to be computed), for example, using the calibration unit 126, the cell concentration of the culture medium in the first circuit 136 at the time of step S25. The method may include, at step S31, computing (or causing to be computed), for example, using the calibration unit 126 the cell concentration based on the information (e.g., volume and number of cells of the first circuit 136) stored in the memory unit 122. This cell concentration may be defined as a first concentration C1. The method may include, in step S31, associating (or causing to be associated), for example, using the calibration unit 126, the first concentration C1 with the first measurement value M1. The method may include, in step S31, calculating (or causing to be calculated), for example, using the calibration unit 126, the cell concentration of the culture medium in the second circuit 138 at the time of step S29. The method may include, in step S31, computing (or causing to be computed), for example, using the calibration unit 126, the cell concentration based on the information (e.g., volume and number of cells of the second circuit 138) saved in the memory unit 122. This cell concentration may be defined as a second concentration C2. The method may include, in step S31, associating (or causing to be associated), for example, using the calibration unit 126, the second concentration C2 with the second measurement value M2. The cell concentration and the turbidity may have a linear relationship. The method may include, in step S31, creating (or causing to be created), for example, using the calibration unit 126, the calibration curve 140 of the cell concentration and the turbidity (measurement value) from the first concentration C1, the first measurement value M1, the second concentration C2, and the second measurement value M2. The method may include, in step S31, storing (or causing to be stored), for example, using the calibration unit 126, the calibration curve 140 in the memory unit 122. In at least one example embodiment, the first concentration C1 may be computed at the time of step S25, that is, when the memory unit 122 saves the first measurement value M1. In at least one example embodiment, the second concentration C2 may be calculated at the time of step S29, that is, at the time when the memory unit 122 saves the second measurement value M2.

In the instance of the method as illustrated in FIG. 13, more accurate calibration may be performed by measuring the minimum concentration at the time of cell input and the maximum concentration in the cell culture device 10.

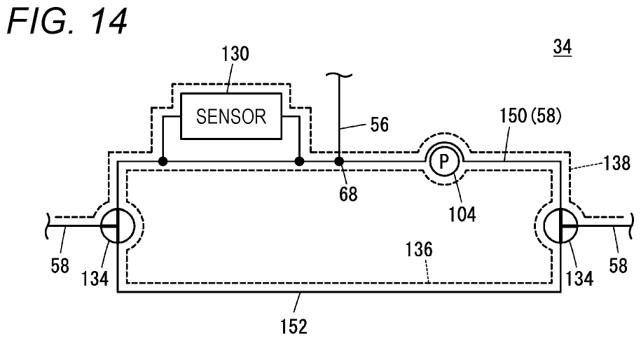

FIG. 14 is a schematic of another example configuration of the sensor unit 34.

In the sensor unit 34 as illustrated in FIG. 11, the sensor 130 may be connected to the sensor flow path 150 in series. In the sensor unit 34 as illustrated in FIG. 14, the sensor 130 may be connected to the sensor flow path 150 in parallel. The sensor unit 34 as illustrated in FIG. 14 may be substantially the same as the sensor unit 34 as illustrated in FIG. 11 except that the sensors 130 are connected in parallel.

As illustrated in FIG. 14, the first circuit 136 may be formed by a closed circuit that includes the sensor flow path 150, the concentration flow path 152, the sensor 130, and the first circulation pump 104. As illustrated in FIG. 14, the second circuit 138 may be formed by a closed circuit that includes the first circulation flow path 58 (which includes the sensor flow path 150), the sensor 130, and the bioreactor 30.

When the sensor 130 is connected to the sensor flow path 150 in parallel as illustrated in FIG. 14, the flow rate of the culture medium flowing through the sensor flow path 150 may not be affected by an increase in internal pressure in the internal flow path and the circuit of the sensor 130. Therefore, the sensor unit 34 in the instance of FIG. 14, a large volume of the cell fluid and the culture medium can flow through the sensor flow path 150 (first circulation flow path 58), while also achieving the same effects as in the instance of the sensor unit 34 as illustrated FIG. 11.

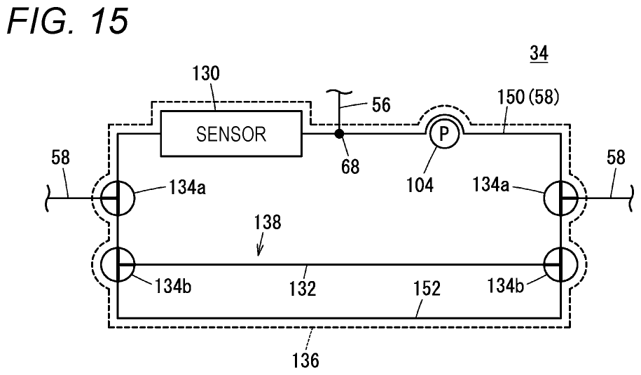

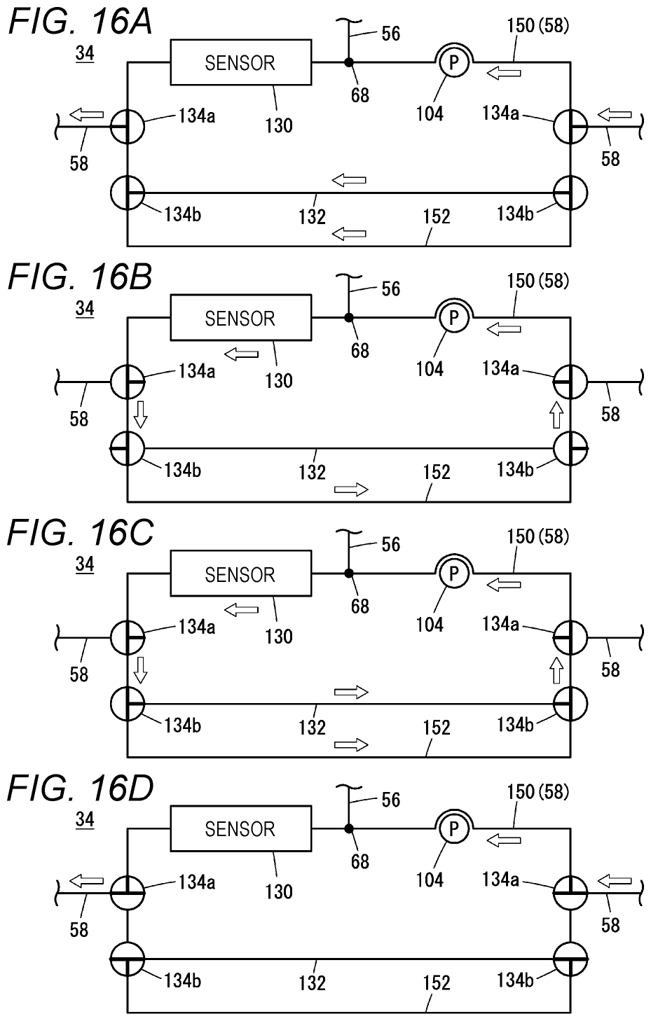

FIG. 15 is a schematic of another example configuration of the sensor unit 34. FIGS. 16A-16D are schematics illustrating a connection state between the first circulation flow path 58, the sensor flow path 150, the concentration flow path 152, and the dilution flow path 132 in the sensor unit 34 as illustrated in FIG. 15. The sensor unit 34 as illustrated in FIG. 15 includes the sensor flow path 150, the sensor 130, the concentration flow path 152, the dilution flow path 132, two first valves 134a, and two second valves 134b.

In the instance of FIG. 15, the calibration processing may be performed using a closed circuit that does not include the bioreactor 30. The dilution flow path 132 may be a tube member. The dilution flow path 132 may be connected to the concentration flow path 152 in parallel. The volume of the sensor flow path 150, the volume of the concentration flow path 152, and the volume of the dilution flow path 132 may be stored in the memory unit 122 in advance. The first valve 134a may be disposed at each connection point between the sensor flow path 150 and the concentration flow path 152. The second valve 134b may be disposed at each connection point between the concentration flow path 152 and the dilution flow path 132.

In the instance of FIG. 15, the two first valves 134a and the two second valves 134b may be, for example, three-way valves. As illustrated in FIGS. 16A-16D, both of the two first valves 134a and the two second valves 134b may be configured to switch between a communication state and a cutoff state of the sensor flow path 150, the concentration flow path 152, and the dilution flow path 132.

In the instance of FIG. 15, the first circuit 136 may be formed by a closed circuit including the sensor flow path 150, the concentration flow path 152, the sensor 130, and the first circulation pump 104. Furthermore, in the instance of FIG. 15, the second circuit 138 may be formed by the dilution flow path 132 connected to the first circuit 136 in parallel.

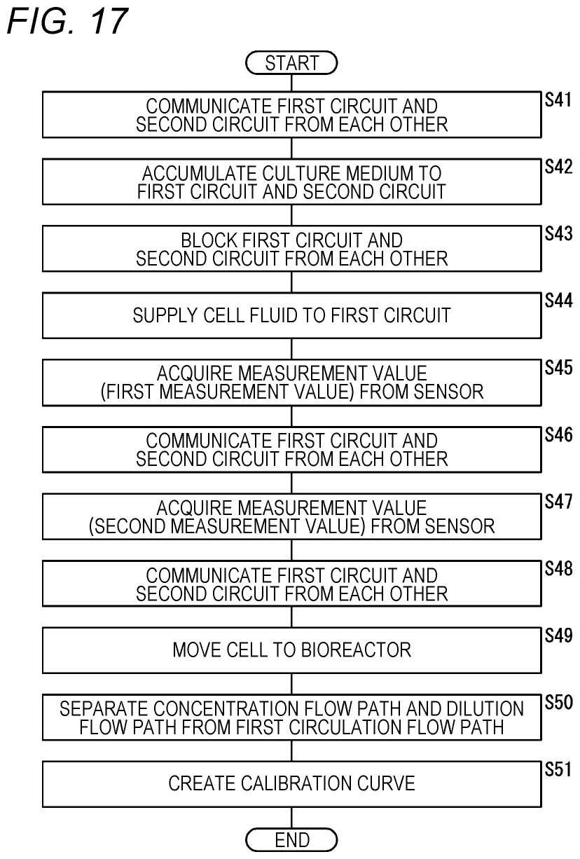

FIG. 17 is a flowchart illustrating another example calibration method. Before the calibration processing, such as illustrated in FIG. 17, a user or operator of the cell culture device 10 receives, or counts, the number of cells in the cell fluid, for example, from or using a cell counter or the like. The user or operator may fill a medical bag or container with the cell fluid for which the number of cells has been counted. The user or operator may set the medical bag or container including the cell fluid for which the number of cells has been counted, or is known, in the supply unit 22. The user or operator may fill the medical bag or container with a culture medium that does not include cells. The user or operator may set the medical bag or container including the culture medium in the supply unit 22. The user or operator may enter the counted or known number of cells into the memory unit 122 of the cell culture device 10. The user or operator may fill the culture medium in the medical bag or container. The user or operator may set the medical bag or the container in the supply unit 22.

The method may include, in step S41, controlling, for example, using the control unit 124, the first valve 134a and the second valve 134b. The method may include, in step S41, causing, for example, using the control unit 124, a portion of the first circulation flow path 58 other than the sensor flow path 150 and the sensor flow path 150 to communicate with each other. The method may include, in step S41, causing, for example, using the control unit 124 the first circulation flow path 58 and the concentration flow path 152 to communicate with each other. The method may include, in step S41, causing, for example, using the control unit 124, the first circulation flow path 58 and the dilution flow path 132 to communicate with each other. The method may include, in step S41, causing, for example, using the control unit 124, the sensor flow path 150 and the concentration flow path 152 to communicate with each other. The method may include, in step S41, causing, for example, using the control unit 124 the sensor flow path 150 (and the concentration flow path 152) and the dilution flow path 132 to communicate with each other.

The method may include, in step S42, connecting (or causing to be connected), for example, using the control unit 124, the first supply flow path 56 to the medical bag or container of the culture medium. The method may include, in step S42, operating, for example, using the control unit 124, the first supply pump 102 and the first circulation pump 104. According to the operations of the first supply pump 102 and the first circulation pump 104, the supply unit 22 may supply the culture medium containing no cells to the first circulation flow path 58. As a result, the culture medium not containing cells may accumulate in both the first circuit 136 and the second circuit 138. After supplying the culture medium, the method may include, stopping (or causing to be stopped), for example, using the control unit 124, the first supply pump 102.

The method may include, in step S43, controlling, for example, using the control unit 124, the first valve 134a and the second valve 134b. The method may include blocking (or causing to be blocked), for example, using the control unit 124 a portion of the first circulation flow path 58 other than the sensor flow path 150 from the sensor flow path 150. The method may include, in step S43, blocking (or causing to be blocked), for example, using the control unit 124, a portion of the first circulation flow path 58 other than the sensor flow path 150 from the concentration flow path 152. The method may include, in step S43, blocking (or causing to be blocked), for example, using the control unit 124, a portion of the first circulation flow path 58 other than the sensor flow path 150 and the dilution flow path 132 from each other. The method may include, in step S43, causing, for example, using the control unit 124, the sensor flow path 150 and the concentration flow path 152 to communicate with each other. The method may include, in step S43, blocking (or causing to be blocked), for example, using the control unit 124, the sensor flow path 150 (and the concentration flow path 152) and the dilution flow path 132 from each other. In this manner, the first circuit 136 may be formed, and the first circuit 136 and the second circuit 138 may be separated from each other.

The method may include, in step S44, connecting (or causing to be connected), for example, using the control unit 124, the first supply flow path 56 to the medical bag or container of the cell fluid. The method may include, in step S44, operating, for example, using the control unit 124, the first supply pump 102 and the first circulation pump 104. The supply unit 22 may supply the cell fluid to the sensor flow path 150 in accordance with the operations of the first supply pump 102 and the first circulation pump 104. In this manner, the first circulation flow path 58 other than the sensor flow path 150 may be blocked from the sensor flow path 150. Therefore, in the sensor unit 34, the cell fluid may be supplied only to the first circuit 136. The method may include, in step S44, stopping (or causing to be stopped), for example, using the control unit 124, the first supply pump 102 after the supply of the cell fluid is completed.

The method may include, in step S45, acquiring, for example, using the calibration unit 126, the measurement value from the sensor 130. This measurement value may be referred to as a first measurement value M1. The memory unit 122 may save the first measurement value M1.

The method may include, in step S46, causing, for example, using the control unit 124, the sensor flow path 150 (and the concentration flow path 152) and the dilution flow path 132 to communicate with each other by controlling the second valve 134b. As a result, the first circuit 136 and the second circuit 138 may communicate with each other. The method may include, in step S46, mixing (or causing to be mixed), the culture medium of the second circuit 138 and the culture medium of the first circuit 136. In this manner, the culture medium containing the cells may be diluted.

The method may include, in step S47, acquiring, for example, using the calibration unit 126, the measurement value from the sensor 130. This measurement value may be referred to as a second measurement value M2. The memory unit 122 may store the second measurement value M2.

The method may include, in step S48, controlling, for example, using the control unit 124, the first valve 134a and the second valve 134b. The method may include, bring, for example, using the control unit 124, the respective flow paths into the same communication state as in step S1.

The method may include, in step S49, moving (or causing to be moved, for example, using the control unit 124, the cells in the sensor unit 34 to the bioreactor 30. As in step S8 as illustrated in FIG. 5, the method may include in step S49, operating, for example, using the control unit 124, the first supply pump 102 and the first circulation pump 104 to supply the culture medium not containing cells from the supply unit 22 to the first circulation flow path 58.

The method may include, in step S50, controlling, for example, using the control unit 124, the first valve 134a and the second valve 134b. The method may include, in step S50, causing, for example, using the control unit 124, a portion of the first circulation flow path 58 other than the sensor flow path 150 and the sensor flow path 150 to communicate with each other. The method may include, in step S50, blocking (or causing to be blocked), for example, using the control unit 124, the first circulation flow path 58 and the concentration flow path 152 from each other. The method may include, in step S50, blocking (or causing to be blocked), for example, using the control unit 124, the first circulation flow path 58 and the dilution flow path 132 from each other. As a result, the concentration flow path 152 and the dilution flow path 132 may be separated from the first circulation flow path 58.

The method may include, in step S51, creating, for example, using the calibration unit 126, the calibration curve 140. The method may include, in step S51, computing (or causing to be computed), for example, using the calibration unit 126, the cell concentration of the culture medium in the first circuit 136 at the time of step S45. The method may include, in step S51, computing (or causing to be computed), for example, using the calibration unit 126, the cell concentration based on the information (e.g., volume and number of cells of the first circuit 136) stored in the memory unit 122. This cell concentration may be defined as a first concentration C1. The method may include, in step S51, associating (or causing to be associated), for example, using the calibration unit 126, the first concentration C1 with the first measurement value M1. The method may include, in step S51, calculating (or causing to be calculated), for example, using the calibration unit 126, the cell concentration of the culture medium in the second circuit 138 at the time of step S47. The method may include, in step S51, computing (or causing to be computed), for example, using the calibration unit 126, the cell concentration based on the information (e.g., volume of the first circuit 136, the volume of the second circuit 138, and number of cells of circuits) stored in the memory unit 122. This cell concentration may be defined as a second concentration C2. The method may include, in step S51, associating (or causing to be associated), for example, using the calibration unit 126, the second concentration C2 with the second measurement value M2. The cell concentration and the turbidity may have a linear relationship. The method may include, in step S51, creating, for example, using, the calibration unit 126, the calibration curve 140 of the cell concentration and the turbidity (measurement value) from the first concentration C1, the first measurement value M1, the second concentration C2, and the second measurement value M2. The method may include, in step S51, storing, for example, using calibration unit 126, the calibration curve 140 in the memory unit 122. In at least one example embodiment, the first concentration C1 may be computed at the time of step S45, that is, when the memory unit 122 stores the first measurement value M1. In at least one example embodiment, the second concentration C2 may be calculated at the time of step S47, that is, at the time when the memory unit 122 saves the second measurement value M2.

In the instance of FIG. 17, the calibration processing may be performed in a short time because of the absence of the bioreactor 30 in the first and second circuits 136, 138.

In at least one example embodiment, the present disclosure provides a cell culture device 10 that may include a cell culture circuit 16 that is configured to circulate a culture medium which includes a cell fluid, a sensor 130 that is disposed in the cell culture circuit and that measures turbidity of the cell fluid, and a calibration unit 126 that calibrates a measurement value of the sensor to a cell concentration of the culture medium. The sensor may measure, as a first measurement value (M1), the turbidity of a first volume of the culture medium, where the first volume of the culture medium includes a cell fluid that has a predetermined number of cells is supplied. The sensor may measure, as a second measurement value (M2), the turbidity of a second volume of the culture medium, where the second volume of the culture medium is prepared by diluting the first volume of the culture medium with an amount of a culture medium that does not include cells. The calibration unit may calculate the cell concentration of the culture medium in which the cell fluid is supplied as a first concentration (C1). The calibration unit may calculate the cell concentration of the culture medium after dilution as a second concentration (C2). The calibration unit may associate the first concentration with the first measurement value. The calibration unit may associate the second concentration with the second measurement value to create a calibration curve 140 between the measurement value of the sensor and the cell concentration.

In at least one example embodiment, the cell culture device may include a supply unit 22 that supplies the cell fluid to the cell culture circuit and a control unit 124 that controls the cell culture circuit. The cell culture circuit may include a first circuit 136 that forms a closed circuit including the sensor, a second circuit 138 connected in parallel to the first circuit, and a plurality of valves 134, 134a, and 134b disposed at respective connection points between the first circuit and the second circuit. The control unit may control each of the valves to block the first circuit and the second circuit from each other in a state where the first circuit and the second circuit are filled with the culture medium not containing cells, and after the cell fluid containing a predetermined number of cells is supplied to the first circuit by the supply unit, the control unit may control the valve to cause the first circuit and the second circuit to communicate with each other, thereby diluting the culture medium of the first circuit with the culture medium of the second circuit.

In at least one example embodiment, the first circuit may include a bioreactor 30 and a pump 104.

In at least one example embodiment, the first circuit may include the first bioreactor 30 and the pump, and the second circuit may include a second bioreactor 144.

In at least one example embodiment, the sensor may be connected to the closed circuit in parallel.

In at least one example embodiment, the cell culture device may include a sensor flow path 150 in which the sensor is disposed, a supply unit that supplies the cell fluid to the cell culture circuit, and a control unit that controls the cell culture circuit. The cell culture circuit may include a first circuit configured to form a closed circuit including the sensor flow path, a second circuit connected in parallel to the first circuit and configured to form a closed circuit including the sensor flow path, and a plurality of valves disposed at respective connection points between the first circuit and the second circuit. When the first circuit and the second circuit are filled with the culture medium not containing cells, the control unit may control each of the valves to form the first circuit including the sensor flow path and to block the first circuit and the second circuit from each other, and after the cell fluid containing a predetermined number of cells is supplied to the first circuit by the supply unit, the control unit may control the valve to form the second circuit including the sensor flow path and to block the first circuit and the second circuit from each other to dilute the culture medium of the sensor flow path with the culture medium of the second circuit.

In at least one example embodiment, the sensor flow path may include a pump, and the second circuit may include a bioreactor.

In at least one example embodiment, the present disclosure provides a calibration method for a cell culture device. The cell culture device may include a circuit that is configured to circulate a culture medium that includes a cell fluid is supplied, a sensor that is arranged in the cell culture circuit and measures turbidity of the cell fluid, and a calibration unit that calibrates a measurement value of the sensor to a cell concentration of the culture medium. The sensor may measure, as a first measurement value, the turbidity of a first volume of the culture medium, where the first volume of the culture medium includes a cell fluid that includes a predetermined number of cells. The sensor may measure, as a second measurement value, the turbidity of a second volume of the culture medium, where the second volume of the culture medium is prepared by diluting the first volume of the culture medium with an amount of a culture medium that does not include cells. The method may include, for example, using the calibration unit, calculating the cell concentration of the culture medium in which the cell fluid is supplied as a first concentration. The method may include calculating, for example, using the calibration unit, the cell concentration of the culture medium after dilution as a second concentration. The method may include associating, for example, using the calibration unit, the first concentration with the first measurement value. The method may include associating, for example, using the calibration unit, the second concentration with the second measurement value. The method may include creating a calibration curve between the measurement value of the sensor and the cell concentration using the first association and the second association.

1. A cell culture device comprising:
a cell culture circuit configured to circulate a culture medium;
a sensor that is disposed in the cell culture circuit, the sensor configured to measure a turbidity of the cell medium; and
a calibration unit in communication with the sensor, the calibration unit configured to prepare a calibration curve and, using that calibration curve, to calibrate a measurement value as acquired by the sensor.

2. The cell culture device of claim 1, wherein
the sensor is configured to measure, as a first measurement value, a first turbidity of a first volume of the culture medium, the first volume of the culture medium including a cell fluid, the cell fluid having a predetermined number of cells;
the sensor is configured to measure, as a second measurement value, a second turbidity of a second volume of the culture medium, the second volume of the culture medium being the first volume of the culture medium diluted with a third volume of the culture medium, the third volume of the culture medium not including cells;
the calibration unit is configured to calculate, as a first concentration, a first cell concentration of the first volume of the culture medium;
the calibration unit is configured to calculate, as a second concentration, a second cell concentration of the second volume of the culture medium; and
the calibration unit is configured to create the calibration curve by associating the first concentration with the first measurement value and associating the second concentration with the second measurement value.

3. The cell culture device of claim 1, wherein the cell culture device further comprises:
a supply unit that supplies the cell medium to the cell culture circuit.

4. The cell culture device of claim 1, wherein the cell culture device further comprises:
a control unit in communication with at least one of the cell culture circuit, the sensor, and the calibration unit.

5. The cell culture device of claim 1, wherein the cell culture circuit includes:
a first circuit that forms a closed circuit including the sensor; and
a second circuit connected to the first circuit in parallel.

6. The cell culture device of claim 5, wherein the cell culture circuit further includes:
a valve disposed at a connection point between the first circuit and the second circuit.

7. The cell culture device of claim 6, wherein
the culture medium includes a first volume of the culture medium, a second volume of the culture medium, and a third volume of the culture medium;
the first volume of the culture medium includes a cell fluid, the cell fluid having a predetermined number of cells;
the second volume of the culture medium is the first volume of the culture medium diluted with the third volume of the culture medium, the third volume of the culture medium not including cells;
in a first state, the first circuit and the second circuit include the third volume of the culture medium, the valve disposed between the first circuit and the second circuit is in a closed position blocking the first circuit and the second circuit from each other;
in a second state, the cell fluid is supplied to the first circuit to form the first volume of the culture medium; and
in a third state, the valve disposed between the first circuit and the second circuit is in an opened position to allow the first volume of the culture medium to be diluted by the third volume of the culture medium, the second volume of the culture medium being the first volume of the culture medium diluted with the third volume of the culture medium, the third volume of the culture medium not including containing cells, and to prepare the second volume of the culture medium.

8. The cell culture device of claim 5, wherein
the first circuit includes a bioreactor and a pump.

9. The cell culture device of claim 8, wherein the bioreactor is a first bioreactor, and the second circuit includes a second bioreactor.

10. The cell culture device of claim 5, wherein
the sensor is connected to the closed circuit in parallel.

11. The cell culture device of claim 5, the cell culture device further comprises:
a sensor flow path in which the sensor is disposed,
the first circuit including at least a portion of the sensor flow path, and the second circuit including at least a portion of the sensor flow path.

12. The cell culture device of claim 11, wherein
the sensor flow path includes a pump, and
the second circuit includes a bioreactor.

13. A method for calibrating a cell culture device, the method comprising:
measuring, using a sensor of the cell culture device, a first turbidity of a first volume of a culture medium circulating in a cell culture circuit of the cell culture device, the first volume of the culture medium including a cell fluid, the cell fluid having a predetermined number of cells;
measuring, using the sensor of the cell culture device, a second turbidity of a second volume of the culture medium circulating in the cell culture circuit of the cell culture device, the second volume of the culture medium including no cells;
calculating, using a calibration unit of the cell culture device, a first cell concentration of the first volume of the culture medium;
calculating, using the calibration unit of the cell culture device, a second cell concentration of the second volume of the culture; and
creating a calibration curve for a measurement value of the sensor and a cell concentration by associating the first cell concentration with the first turbidity and associating the second cell concentration with the second turbidity.

14. The method of claim 13, wherein the method further includes:
preparing the first volume of the culture medium.

15. The method of claim 14, wherein the first volume of the culture medium is prepared by contacting the cell fluid to a third volume of the culture medium.

16. The method of claim 13, wherein the method further includes:
preparing the second volume of the culture medium.

17. The method of claim 16, wherein the second volume of the culture medium is prepared by contacting the first volume of the culture medium with a third volume of the culture medium, the third volume of the culture medium not including cells.

18. A method for calibrating a cell culture device, the method comprising:
creating a calibration curve for the cell culture device, the calibration curve created by
associating a first cell concentration of a first volume of a cell medium circulating by the cell culture device with a first turbidity of the first volume of the cell medium as measured by a sensor in the cell culture device, the first cell concentration calculated using a calibration unit of the cell culture device; and
associating a second cell concentration of a second volume of the cell medium circulating by the cell culture device with a second turbidity of the second volume of the cell medium as measured by the sensor in the cell culture device, the second cell centration calculated using the calibration unit of the cell culture device.

19. The method of claim 18, wherein the method further includes:
preparing the first volume of the culture medium by contacting the cell fluid to a third volume of the culture medium.

20. The method of claim 19, wherein the method further includes:
preparing the second volume of the culture medium by contacting the first volume of the culture medium with a fourth volume of the culture medium, the fourth volume of the culture medium not including cells.
